(19) United States
(12) Patent Application Publication
(10) Pub. No.: US 2022/0160612 A1
(43) Pub. Date: May 26, 2022

(54) COSMETIC USE OF AMYLOSE-RICH STARCH AS FILM-FORMING AGENT WITH BARRIER AND FIXATIVE EFFECTS

(71) Applicant: ROQUETTE FRERES, Lestrem (FR)

(72) Inventors: Léon MENTINK, LILLE (FR); Daniel WILS, MORBECQUE (FR); Géraldine LOUVET-POMMIER, LACHELLE (FR); Camille LACORE, MONTMORENCY (FR)

(21) Appl. No.: 17/593,932

(22) PCT Filed: Mar. 27, 2020
(86) PCT No.: PCT/EP2020/058677

§ 371 Date: Sep. 28, 2021

(30) Foreign Application Priority Data
Mar. 29, 2019 (FR) FR1903339

(51) Int. Cl. A61K 8/73; A61Q 17/00; A61Q 19/00; A61Q 5/00

(52) U.S. Cl. CPC A61K 8/732; A61Q 17/00; A61K 2800/40; A61Q 5/00; A61Q 19/00

(57) ABSTRACT

The invention relates to the field of film-forming agents for cosmetic or dermatological use, that are capable of conferring a barrier effect or a fixative effect on a cosmetic preparation. The barrier effect makes it possible to protect the skin from environmental pollution, for example microparticles and volatile organic compounds, and more particularly from urban atmospheric pollution. The fixative effect makes it possible to reduce or eliminate the transfer of pigments and/or dyes, and to increase the hold of the hairstyle or the shaping of head hair, body hair or eyelashes.

### TECHNICAL FIELD

The invention relates to the field of film-forming agents for cosmetic or dermatological use, used to give a preparation for topical use barrier properties to environmental pollution, for example to microparticles and to volatile organic compounds, and more particularly to urban atmospheric pollution, and/or properties for fixing said preparation for topical use.

The present application provides a solution to three current needs in cosmetics: providing a product which is as natural as possible, protecting the skin from environmental attacks using an easy and pleasant cosmetic product for daily use; fixing products for cosmetic use on the skin or skin appendages.

Environmental pollution, and more particularly atmospheric pollution, also known as “smog” or “urban dust”, is composed of particles and inorganic fibers which may comprise heavy metals, and toxic or carcinogenic organic compounds such as polycyclic aromatic hydrocarbon compounds, furans, aldehydes, which may even be associated with pathogenic microorganisms. Such particles have sizes ranging from less than 1 μm up to 500 μm. The smaller these particles, the more toxic they are. According to a study carried out by L'Oréal, particles of size ranging from 2.5 μm to 10 μm are the most harmful: they penetrate deep into the epidermis, where they cause serious chemical degradation.

Atmospheric pollution causes premature skin aging. It has been demonstrated that the process of aging is induced by biological dysfunctions such as those caused by this pollution, for example peroxidation of the lipids of the epidermis, alteration of proteins, abnormal loss of water, and also caused by oxidative stress, apoptosis and cell damage caused by UVB radiation. These dysfunctions cause the appearance of blackheads, a reduction in the skin's radiance, and an increase in the skin's sensitivity.

Protecting the skin from environmental pollution is thus a new target in cosmetics in order to protect the appearance and the health of the skin while maintaining a pleasant texture, from a sensory perspective on the skin, the nails or the hair.

Another consumer requirement is that cosmetic products, particularly those for daily use such as makeup products like foundations, lipsticks, mascaras, or such as hair dyeing products, remain in place once they have been applied. This means that the cosmetic product must remain at least partially, or better still entirely, on the covered area for as long as possible, ideally at least a day, even when the covered area is subject to friction from textiles such as clothing, or even the skin. This non-transfer result is possible due to the presence of a fixative agent in the cosmetic product.

### BACKGROUND ART

Initially, the industry used polymers of natural origin for the film-forming function with an anti-pollution barrier effect. However, the latter had a certain number of disadvantages, especially in terms of color, odor, purity, consistency of efficacy and stability of viscosity. These reasons have led to them being substituted by synthetic or semi-synthetic polymers. In this regard, N-vinylimidazole polymers or copolymers, silicone compounds or silicone gums, and finally carbomers are known, which are highly used in cosmetics. The Carbopol® range developed by Lubrizol is one example thereof. Mention may be made in particular of the product Carbopol® Ultrez 10NF which is a copolymer of polyethylene glycol and long-chain alkyl acid ester, created to give film-forming properties to a wide variety of cosmetic formulations.

For their film-forming function with fixative effect, the industry currently uses synthetic polymers such as acrylate polymers or acrylate and allyl methacrylate copolymers (such as Fixate™ G-100 from Lubrizol), copolymers of N-vinylpyrrolidone and of vinyl acetate, crosslinked methyl methacrylate polymers, polyvinylpyrrolidones, silicones, PEG-40 or PEG-60 hydrogenated castor oils.

According to patent EP1684731, it is known practice to use pregelatinized and fluidized hydroxypropylated pea starch as film-forming agent for coating or film-coating solid pharmaceutical forms such as tablets. This document does not disclose the use of amylose-rich starch, and a fortiori hydroxypropylated pea starch, as film-forming agent in a cosmetic preparation.

### Technical Problem

Nowadays, the cosmetics industry is confronted with new challenges in terms of preserving the environment, conserving fossil resources, carbon footprint and in terms of the health and safety of consumers. From this perspective, the industry is rethinking formulations and returning, where possible, to the use of solutions of natural origin for the formulation of its products. Science and technology are progressing in this field with the aim of proposing reliable and efficient technical solutions which enable manufacturers to achieve film-forming functions which are effective in the products they produce, with natural ingredients which are both environmentally friendly and consumer friendly.

Surprisingly and unexpectedly, the applicant observed that it is possible, for cosmetic purposes, to provide a starch which is easy to use, in particular which is directly dispersible and soluble in cold water, while being stable in the cosmetic preparation. They were also able to observe that such a starch makes it possible, after applying a cosmetic preparation containing same, to create a film with a particularly effective barrier and/or fixative effect.

### OBJECT OF THE INVENTION

A use is proposed of at least one starch, in a preparation for topical use, preferably cosmetic or dermatological use, as film-forming agent with a barrier effect to environmental pollution and/or with a fixative effect, wherein said starch has:

an amylose content of greater than or equal to 30%, preferentially between 30% and 75%,

and a Brookfield viscosity in aqueous dispersion at 25° C. at 20% by weight of solids of between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s.

According to another aspect of the invention, the starch which can be used for the use makes it possible to form, on the epidermis or the skin appendages, a film which protects from environmental pollution due to microparticles suspended in the air and/or volatile organic compounds which are allergenic, such as fragrances, or which are harmful for or toxic to the skin, and preferentially from pollution due to atmospheric microparticles, such as “urban dust”.

According to another aspect of the invention, the starch useful for the use makes it possible to create a barrier to allergenic products of external origin in liquid or volatile form, such as fragrances, essential oils, organic solvents, or in the form of solid particles as for example pollens.

According to another aspect of the invention, the starch useful for the use makes it possible to form a film with fixative effect, which makes it possible to increase the hold of the preparation for topical use, especially pigments, dyes, hair dyeing products, or which makes it possible to increase the hold of the hairstyle or shaping of the head hair, body hair or eyelashes, or which makes it possible to give a non-transfer property to the preparation for topical use, for example the non-transfer of makeup products.

According to another aspect of the invention, the starch useful for the use according to the invention is a leguminous plant starch which is native and/or thermally modified and/or chemically modified.

According to another aspect of the invention, the at least one leguminous plant starch is the only amylaceous or of amylaceous origin film-forming agent.

According to another aspect, a preparation for topical use is proposed, preferably for cosmetic or dermatological use, comprising at least one such starch, preferably a leguminous plant starch.

### BRIEF DESCRIPTION OF THE DRAWINGS

FIG. 1 shows the photographs of the untreated skin area, denoted control, and the skin area covered with a film formed by the emulsion with reference A, before application of the emulsions (T0), after application of the emulsions and the charcoal powder (T2), and after rinsing with water (T3).

FIG. 2 shows the photographs of the skin area covered with a film formed by the emulsion with reference B, and the skin area covered with the emulsion according to the invention INV1, before application of the emulsions (T0), after application of the emulsions and the charcoal powder (T2), and after rinsing with water (T3).

FIG. 3 shows the photograph of the tissue papers during the test of non-transfer of the foundation according to the invention.

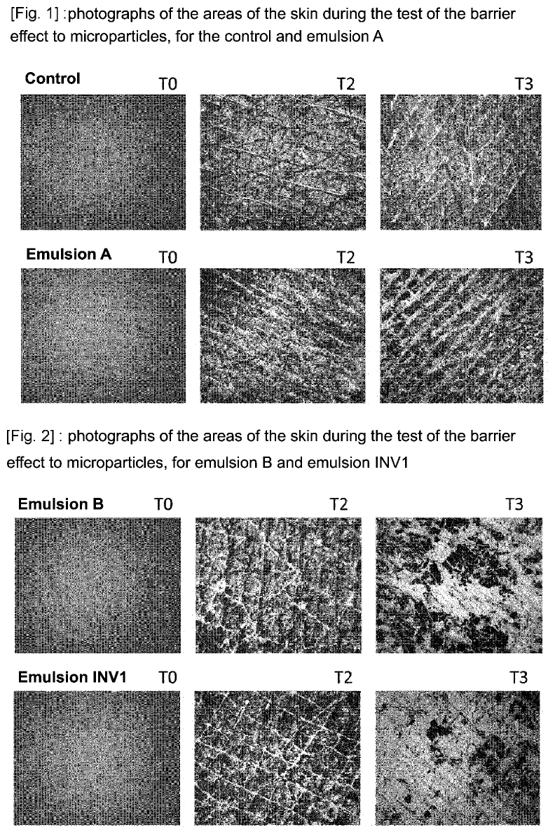

### DESCRIPTION OF EMBODIMENTS

A use is proposed of at least one starch, in a preparation for topical use, as film-forming agent with a barrier effect to environmental pollution and/or with a fixative effect, wherein said starch has:

an amylose content of greater than or equal to 30%, preferentially between 30% and 75%,

and a Brookfield viscosity in aqueous dispersion at 20% by weight of solids and at 25° C. of between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s.

Starch Useful for the Invention

The two essential characteristics of the starch useful for the invention are that it has an amylose content of greater than or equal to 30% and a Brookfield viscosity in aqueous dispersion at 25° C. at 20% by weight of solids of between 10 and 10,000 mPa·s.

Within the meaning of the present invention, the viscosity is a Brookfield viscosity determined for example by means of a Brookfield RDVD-I+ viscometer (Brookfield Engineering Laboratories, INC. Middleboro, Mass., USA) using one of the spindles with the references RV1, RV2, RV3, RV4, RV5, RV6 or RV7 and without using the equipment referred to as “Helipath Stand”. The rotation of the spindle is set at 20 revolutions per minute. The spindle, from RV1 to RV7, is selected such that the displayed viscosity value is between 10% and 100% of the total scale of viscosity possible with said spindle, as indicated by the manufacturer. In order to carry out this viscosity measurement, 300 ml of an aqueous suspension or aqueous solution containing 20% by weight of starch solids prepared at 25° C. with mechanical stirring, for example with a deflocculating stirrer blade at 250 rpm for 15 minutes, are placed in a 400 ml low-form beaker (diameter of approximately 7.5 cm). The viscosity value is taken at the end of the 3rd rotation. The measurement is carried out according to all the manufacturer's recommendations in order to obtain a reliable viscosity measurement, for example in the manual “Operating Instructions, Manual No. M/92-021-M0101, Brookfield Digital Viscometer, Model DV-I+”.

In particular, the amylose content is comprised within a range extending from 30% to 75%, preferably from 30% to 45%, and further preferable from 35% to 42%. The amylose percentages are expressed as dry weight relative to the dry weight of starch, and determined before any subsequent treatment such as hydrolysis and/or alkylation of said starch.

The Brookfield viscosity in aqueous dispersion at 25° C. at 20% by weight of solids is between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s. These Brookfield viscosity ranges may be combined with the amylose content ranges.

According to a first embodiment of the use according to the invention, it is proposed to use a leguminous plant starch which is native or chemically modified.

Native or Chemically Modified Leguminous Plant Starch

Within the meaning of the present invention, “leguminous plant” means any plant belonging to the families of the cesalpiniaceae, mimosaceae or papilionaceae, and particularly any plant belonging to the family of the papilionaceae, for example pea, bean, broad bean, faba bean, lentil or lupin.

Thus, the leguminous plant starch useful for the use according to the invention may be selected from starches of peas, of chickpeas, of broad beans, of faba beans, of beans, of lupins, of lentils, or of lupin. Preferentially, the leguminous plant starch is selected from pea starches, and is most preferentially a starch of Pisum sativum.

The leguminous plant starch may be selected from those which are native or those which are chemically modified. The chemically modified leguminous plant starch is a leguminous plant starch which has undergone at least one of the following chemical modifications: hydroxyalkylation, carboxyalkylation, succinylation, alkylation, acetylation, cationization, anionization. These chemical modifications are modifications for stabilizing the leguminous plant starch, in other words for stabilizing the viscosity in aqueous solution, in that they may it possible to reduce or eliminate retrogradation of a gel or of an aqueous solution of said starch. Thus, the chemically modified leguminous plant starch may be selected from hydroxyalkylated, carboxyalkylated, octenylsuccinylated, succinylated, or acetylated leguminous plant starches. Such starches are generally referred to as “stabilized starches” since they have a reduced or controlled, or no, tendency to retrogradation.

The hydroxyalkylated leguminous plant starch useful for the invention may be a hydroxyalkylated leguminous plant starch having a content of hydroxyalkyl groups ranging from 0.1 to 20% by dry weight relative to the dry weight of hydroxyalkylated starch, preferentially from 1 to 10%.

Preferentially, the leguminous plant starch is selected from hydroxypropylated, hydroxyethylated or carboxymethylated leguminous plant starches, and is most preferentially a hydroxypropylated leguminous plant starch.

Within the meaning of the invention, “hydroxypropylated leguminous plant starch” is intended to mean a leguminous plant starch substituted with hydroxypropyl groups by any technique known to those skilled in the art, for example by etherification reaction with propylene oxide. Within the context of the invention, a hydroxypropylated leguminous plant starch preferably has a content of hydroxypropyl groups of between 0.1 and 20% by dry weight relative to the dry weight of hydroxypropylated starch, preferentially between 0.5 and 15% by weight, more preferentially between 1 and 10% by weight, even more preferentially between 5 and 9% by weight, and most preferentially close to 7% by weight. In particular, this content is determined by proton Nuclear Magnetic Resonance spectrometry, in particular according to the EN ISO 11543:2002 F standard.

According to a second embodiment, it is proposed to use a fluidized, dextrinized or hydrolyzed leguminous plant starch. According to a third embodiment, the leguminous plant starch is selected from acetylated starches.

Fluidized Leguminous Plant Starch

“Fluidized leguminous plant starch” is intended to mean a granular starch which has undergone a mild acid treatment in dry or anhydrous medium, at low temperature, generally less than 100° C., preferentially less than 80° C. The fluidization does not cause any modification to the molecular weight of the starch, but rather a destructuring of the intermolecular bonds between the macromolecules of amylose and amylopectin, such that the viscosity of a solution of such a starch is reduced compared to a non-fluidized starch.

Hydrolyzed Leguminous Plant Starch

Within the meaning of the present invention, “hydrolyzed leguminous plant starch” is intended to mean a leguminous plant starch which has undergone a hydrolysis operation, that is an operation which aims to reduce the average molecular weight thereof. The person skilled in the art knows how to obtain such starches, for example by chemical treatments such as oxidation and acid treatments, or also by enzymatic treatments. The hydrolysis is generally carried out on a gelatinized or liquefied starch. Those skilled in the art will naturally adjust the level of hydrolysis based on the viscosity desired for the starch.

Dextrinized Leguminous Plant Starch

“Dextrinized leguminous plant starch” is intended to mean a leguminous plant starch which has undergone a dextrinization operation. Dextrinization is a hydrolysis of a starch powder carried out in dry or anhydrous medium. Dextrinization is a process for modifying starch in the powder state which uses the combined action of heat and, generally, a chemical agent capable of hydrolyzing the glycosidic bonds. Whether discontinuous or continuous, these methods call for transformation temperatures of greater than 100° C. and the optional presence of an acid, an alkaline agent and/or an oxidizer, in a dry medium or one with a low moisture content, generally less than 25% m, or even 15% m. Like hydrolysis, dextrinization makes it possible to reduce the molecular weight

Within the context of the invention, the hydrolyzed or dextrinized leguminous plant starch preferably has a weight-average molecular weight ranging from 1 to 2000 kDa, preferably from 10 to 1000 kDa, most preferentially from 20 to 1000 kDa, and even more preferentially from 100 to 1000 kDa. For example, the molecular weight can range from 200 to 800 kDa, from 200 to 500 kDa, from 200 to 400 kDa, or even from 200 to 300 kDa. The weight-average molecular weight is determined by HPSEC-MALLS (high-performance size exclusion chromatography coupled in-line with multi-angle laser light scattering detection).

According to a preferred variant of this embodiment of the use according to the invention, the modified leguminous plant starch is a hydrolyzed and hydroxyalkylated leguminous plant starch. A most preferred variant is a hydrolyzed and hydroxypropylated leguminous plant starch.

According to a third embodiment, it is proposed to use a thermally modified leguminous plant starch.

Thermally Modified Leguminous Plant Starch

The leguminous plant starch useful for the invention may be a thermally modified leguminous plant starch. These thermal modifications are physical modifications and are those selected from the operations of gelatinization, pregelatinization, extrusion, atomization or drying.

The thermal modifications given above make it possible to increase the aqueous solubility of the leguminous plant starch, or even to make it totally water-soluble. In particular, the starch according to the invention may preferably be made soluble. It can be made soluble by any technique known to those skilled in the art, especially by heat and/or mechanical treatment, for example by an operation of cooking in aqueous medium, which may range from pregelatinization to gelatinization or complete solubilization, optionally followed by a drying step when it is desired to obtain a pulverulent product.

The operation to make the starch soluble may be carried out entirely indifferently before or after the chemical modification and/or the hydrolysis of the starch, or else after the introduction thereof into the preparation for topical use, for example by cooking the preparation for topical use at the time it is produced.

### Combinations of the Embodiments

The starch useful for the invention may be a starch having at least two characteristics selected from: being a leguminous plant starch, being a hydrolyzed starch, being a dextrinized starch, being a fluidized starch, being a thermally modified starch, being a chemically modified starch.

The starch may thus be a hydroxyalkylated, and fluidized or hydrolyzed, and pregelatinized, leguminous plant starch. Preferentially, such a leguminous plant starch will be a hydroxypropylated, hydrolyzed and pregelatinized pea starch.

When the leguminous plant starch is alkylated or hydroxyalkylated, and hydrolyzed, it will preferably be non-granular. It will be advantageously rendered soluble in water by any known technique so that the film-forming composition has very good film-forming properties.

Pregelatinized, hydrolyzed and hydroxypropylated pea starches suitable for the use according to the invention are commercially available and are sold by the Applicant under the brand name Lycoa®, for example Lycoat® RS720 or Lycoat® RS780.

According to another embodiment of the use according to the invention, it is proposed to use an amylose-rich cereal or tuberous plant starch.

Amylose-Rich Cereal or Tuberous Plant Starches

Cereal or tuberous plant starches useful for the invention may be selected from amylose-rich cereal or tuberous plant starches, that is those having an amylose content of greater than or equal to 30%, preferentially between 30% and 75%.

Amylose-rich cereals or tuberous plants are cereals or tuberous plants which have been selected by cross-breeding or hybridization, or which have been genetically modified, to increase the amylose content in their starch.

A known example of amylose-rich maize is amylomaize.

As for the leguminous plant starches described above, the amylose-rich cereal or tuberous plant starches useful for the invention may be native or modified. The modifications may be similar to those described for the above leguminous plant starches, namely thermal modifications such as pregelatinization, atomization, and/or chemical modifications such as hydroxyalkylations, acetylations, cationizations, anionizations, carboxyalkylations. All the embodiments mentioned for a leguminous plant starch can be applied to the amylose-rich cereal or tuberous plant starches.

Optional Additional Modifications

Aside from the thermal and chemical modifications described above, the starch according to the invention may also have undergone one or more other physical and/or chemical modifications, as long as said modifications do not interfere with the desired properties of the starch for the use according to the invention. An example of chemical modification particularly is cross-linking.

Other additional modifications which may be applied to the starch are treatment operations by microwaves or ultrasounds, plasticization or granulation.

Preparation for Topical Use

Within the meaning of the present invention, “preparation for topical use” is intended to mean any composition intended to be brought into contact with the human or animal, preferentially human, skin. It may thus be a cosmetic composition, a dermatological composition, a pharmaceutical composition or a veterinary composition.

The preparation for topical use may comprise contents by weight of film-forming starch according to the invention ranging from 0.6% to 50% by weight relative to the whole preparation for topical use, preferentially of 2% to 30% by weight, more preferentially of 5% to 15%, and most preferentially of approximately 10%.

The content by weight of starch according to the invention may advantageously be high, without any notable harmful impact on the texture of the preparation for topical use, particularly by using a starch according to the invention having a low viscosity, especially a Brookfield viscosity ranging from 50 mPa·s to 1000 mPa·s in aqueous solution at 20% by weight of solids at 25° C.

These preparation for topical use may comprise, aside from the starch described previously, other ingredients commonly used in preparations for topical use, for example water, humectants, emulsifiers, surfactants, thickeners, gelling agents, lubricants, emollients, fatty substances, and particularly also cosmetic or dermatological active agents, and adjuvants such as preservatives, solubilizers or fragrances. Among the cosmetic active agents, it may comprise moisturizing agents such as isosorbide, betaine, glycerine or acetamidoethoxyethanol or else many other active products or products with sensory effects in relation to the skin, for example starch other than the specific starch described in the present application.

Non limiting examples of preparations for topical use comprise lotions, creams, serums, gels, ointments, balms, liquid soaps or shower gels, shampoos, mousses, foundations, antiperspirants and deodorants.

The preparation for topical, preferably cosmetic, use is selected from skincare products, preferentially selected from day creams, suncreams, aftersun creams, self-tanners, masks; haircare products, preferentially selected from shampoos, conditioners in cream or mask or lotion form, hairstyling products in spray or gel or wax form, dyeing products; makeup products, preferentially selected from foundations, eyeshadows, mascaras, nail varnishes, lipsticks, pressed lip colors, lip glosses, eyeliners; hygiene products, preferentially selected from cleansing gels, cleansing wipes or makeup removing wipes, or aqueous-alcoholic solutions or gels.

According to one embodiment, the starch useful for the invention is the only amylaceous film-forming agent in the preparation for topical use.

Film with Barrier Effect to Environmental Pollution

The use of a starch, preferably a leguminous plant starch, according to the invention enables the preparation for topical use to form a film on the surface of the skin or head hair or body hair, which forms a barrier to environmental pollutions which are harmful or toxic for the skin. This film protects the skin, the head hair or the body hair from environmental pollution due to microparticles and to allergenic volatile organic compounds suspended in the air, in particular that of atmospheric microparticles, for example dust known under the name “urban dust”, or plant pollen. This film also protects from pollutants or allergenic substances intentionally applied to the skin, such as perfumes. It makes it possible to reduce or prevent atmospheric microparticles suspended in the air, and/or organic compounds which are volatile or associated with microparticles, from coming into contact with the skin.

Regarding environmental pollution due to microparticles, the barrier film obtained according to the invention makes it possible to reduce the adhesion of said microparticles on the skin. This results in the skin not being in contact with these microparticles, and them therefore not being able to harm the skin's health. The skin is therefore protected from the metabolic degradations usually caused by microparticles.

Regarding environmental pollution due to volatile organic substances or substances attached to microparticles, the barrier film according to the invention makes it possible to prevent said organic substances from diffusing to the skin or to the head hair or to the body hair.

Indeed, the applicant has observed that a preparation for topical use comprising a starch according to the invention enables the partial cell protection of the cells of the epidermis and keratinocytes. “Cell protection” is intended to mean that the use makes it possible to protect the cells of the epidermis, that is to reduce the mortality thereof, when they are exposed to micropollutants such as urban dust and heavy metals such as arsenic, cadmium, cobalt, chromium, nickel, lead, strontium and antimony. In other words, the use according to the invention makes it possible to keep a portion, or even a large amount, preferentially at least 50%, of the cells of the epidermis alive when they are subjected to pollution stresses.

In the absence of a cell protection according to the invention, the cells of the epidermis and the keratinocytes are virtually all killed by exposure to urban dust and to heavy metals. In the presence of a barrier film formed according to the invention, the cells of the epidermis and the keratinocytes are less affected by these pollutants, and thus exhibit a cell mortality reduced by at least 60% compared to exposure in the absence of such a barrier film.

Film with Fixative Effect

The applicant intends “film with fixative effect” to mean that the film formed by the preparation for topical use on a part of the skin or the skin appendages of an individual has adhesion to said part and/or intrinsic mechanical cohesion which are sufficient to withstand mechanical friction from an outside part on the part covered with the film with fixative effect. By way of example of outside part, mention will be made of a skin part of the individual other than the covered part, the skin of another individual, the clothing of the wearing individual or another individual, or wipes. Examples of mechanical friction are for example friction between two cheeks, between a cheek and a hand, or between a cheek and lips, or between two pairs of lips. When the film with fixative effect is rubbed in this way, it stays in place on the skin, retaining its physical integrity and transferring little or no material to the outside part. Following the friction, the outside part comprises only a small amount of, or no, traces of preparation for topical use.

The film with fixative effect prevents the transfer of material from the preparation for topical use, for example from a colored preparation for topical use, from one part of the body to another outside part such as an item of clothing. It also includes reducing running in heat or due to humidity, and therefore good hold of the preparation for topical use after application.

The use of a starch, preferentially a leguminous plant starch, according to the invention as barrier film-forming agent enables the formation of a film with fixative effect. This film with fixative effect makes it possible to reduce or eliminate transfer of the preparations for topical use. In particular, it makes it possible to reduce or eliminate the transfer of pigments and/or dyes from preparations for topical use such as makeup products. This film with fixative effect thus makes it possible to increase the hold of pigments and/or dyes of hair dyeing products. This film with fixative effect also makes it possible to increase the hold of the hairstyle or the shaping of the head hair, body hair or eyelashes.

### EXAMPLES

### Example 1: Preparation of Oil-In-Water (O/W) Emulsions

This example shows the preparation of four oil-in-water (O/W) emulsions according to the compositions of Table 1:

Control Emulsion: emulsion containing only water, oil, the emulsifier “Montanov L” from Seppic, and a preservative, “Sepicide HB” from Seppic,

Emulsion A: according to the prior art, emulsion containing, as film-forming agent, “Keltrol” xanthan gum from CP Kelco,

Emulsion B: according to the prior art, emulsion containing a film-forming mixture, Beauté by Roquette® DS112, consisting of “Keltrol” xanthan gum from CP Kelco, “Natrosol 250 HHR” hydroxyethylcellulose from Hercules, and “Pregeflo CH40” starch from Roquette.

Emulsion INV1: emulsion with a “Lycoat® RS720” pea starch from Roquette Frères used according to the invention.

The preparation procedure for an emulsion is as follows: an aqueous phase is prepared by dispersing the film-forming agent in water at 35-40° C. with stirring with a deflocculating stirrer blade at 1000 revolutions per minute for at least 10 minutes. The emulsifier is then added with stirring at 35-40° C. Separately, the oil is heated to 35-40° C. The oil is then emulsified in the aqueous phase at 35-40° C. with stirring with a deflocculating stirrer blade at 1500 revolutions per minute for 15 minutes. Finally, the preservative is added. The emulsion is kept stirred until it is at ambient temperature.

TABLE 1

| | | | | Emulsion |
|---|---|---|---|---|
| Substance | Control | Emulsion A | Emulsion B | INV1 |
| Helianthus annus | 15% | 15% | 15% | 15% |
| seed oil | | | | |
| Keltrol Xanthan gum | 0 | 0.2% | 0.2% | 0 |
| (CP Kelco) | | | | |
| Pregeflo ® CH40 | 0 | 0 | 3.3% | 0 |
| Starch by Roquette ® | | | | |
| Natrosol 250 HHR | 0 | 0 | 0.5% | 0 |
| Hydroxyethylcellulose | | | | |
| (Hercules) | | | | |
| Lycoate RS720 | 0 | 0 | 0 | 3.8 |
| (Roquette) | | | | |
| Montanov L | 3% | 3% | 3% | 3% |
| Emulsifier (Seppic) | | | | |
| Sepicide HB | 1% | 1% | 1% | 1% |
| Preservative (Seppic) | | | | |
| Water | q.s. 100% | q.s. 100% | q.s. 100% | q.s. 100% |

### Example 2: In Vivo Measurement of the Reduction in the Adhesion of Microparticles

This example compares the performance properties of the anti-pollution by microparticles barrier effects for the four emulsions prepared in example 1.

To this end, tests were carried out on human epidermises from 10 volunteers aged from 18 to 65 years, using as experimental model particles of charcoal sized between 1 μm and 5 μm, termed microparticles. These charcoal microparticles suitably model real polluting microparticles such as exhaust gas particles from combustion engines.

Preparation of the Charcoal Microparticles:

Prior to the tests, a sufficient amount of charcoal microparticles was prepared by subjecting charcoal to the action of a domestic grinder for 10 minutes. This grinding provides particles of micrometric size having a size distribution distributed predominantly between 1 μm and 5 μm.

Procedure for the Test on the Epidermis of the Volunteers:

Four areas of 1 cm by 1 cm were delimited on the forearms of each volunteer. For each area delimited in this way, the following two measurements were carried out: a counting measurement of the number of black microparticles by virtue of a photograph taken with a Dino-Lite digital microscope then processed by image analysis, and a colorimetric measurement using a Minolta© CR-200 chromameter.

Microparticle Count:

The high-resolution photographs taken with the Dino Lite digital microscope are processed using the image processing software GIMP (GNU Image Manipulation Software). Firstly, a projection was carried out along an axis (black and white) to make the black microparticles stand out and to standardize the images. The number of black pixels was then counted with the GIMP software.

Measurement of Colorimetry:

The Minolta CR-200 chromameter is a tool for the objective measurement of surface colors. It provides a result composed of three coordinates, L*, a* and b* in the CIE 1976 color space (also referred to as CIELAB color space). Use was only made of the L* parameter, which characterizes the lightness of the color: L*=0 corresponds to black, L*=100 indicates white.

Carrying Out the Test on Volunteers:

Microparticle counting and colorimetry measurements are carried out before applying the emulsions, that is on clean skins (measurements T0).

Three areas were covered with the emulsions to be tested: one with emulsion A, one with emulsion B, and one with emulsion INV1. To this end, approximately 2 mg of emulsion were applied per cm2 of skin by depositing the required amount with a pipette then by spreading with a spatula. The volunteers then waited 20 minutes to allow the emulsion to dry. The fourth area is left clean in order to form the control area. Measurements were carried out (measurements T1).

The charcoal particles were applied to the four areas by dabbing them on with a makeup sponge impregnated with particles, then measurements were carried out (measurements T2).

The four areas were then rinsed by running 100 ml of water over the whole surface of each area, then measurements were carried out (measurements T3).

Table 2 below tallies the mean number of black pixels.

TABLE 2

| Time of | | | | |
|---|---|---|---|---|
| measurements | Control | Emulsion A | Emulsion B | Emulsion INV1 |
| T0 | 187289 | 199232 | 259063 | 244844 |
| T2 | 628500 | 628872 | 691137 | 813301 |
| T3 | 609106 | 509695 | 589862 | 477910 |

Table 3 gives the measurements of the mean L* parameter.

TABLE 3

| Time of | | | | |
|---|---|---|---|---|
| measurements | Control | Emulsion A | Emulsion B | Emulsion INV1 |
| T0 | 62.07 | 61.84 | 62.16 | 62.8 |
| T2 | 15.72 | 20.11 | 20.27 | 21.02 |
| T3 | 18.64 | 36.15 | 35.86 | 40.92 |

Table 4 indicates the mean variations between T3 and T2 (expressed as %).

TABLE 4

| | Control | Emulsion A | Emulsion B | Emulsion INV1 |
|---|---|---|---|---|
| Mean number | −4.4% | −27.7% | −23.4% | −59.4% |
| of black pixels | | | | |
| Mean L* | +19% | +80% | +77% | +95% |
| parameter | | | | |

When the skin was covered with a film formed from an emulsion INV1 comprising the starch according to the invention, washing with water following exposure to charcoal microparticles, makes it possible to reduce the number of black pixels by 59% and to increase the L* parameter by 95%. This represents a reduction in the number of pixels superior by 32%, and an increase in the L* parameter superior by 15 to 18% to the reference emulsions A and B. The reduction in the number of black pixels is the direct result of the reduction in the number of black particles which have adhered to the skin. The increase in the L* parameter reflects an increase in the lightness of the skin, which results from the reduction in the number of particles which have adhered to the skin. The starch used according to the invention therefore does indeed make it possible to effectively reduce the adhesion of microparticles on the skin.

### Example 2: In Vitro Measurement of Cell Protection

In this example, the ability of emulsions A and INV1 from example 1 to protect cells of the epidermis from the lethal effects of micropollutants is determined by an in vitro test. This in vitro test consists in comparing the cell viability on reconstructed epidermis samples when they are exposed to a mixture of micropollutants, without or with the prior application of an emulsion to the reconstructed epidermis sample.

Table 6 presents the test conditions for six reconstructed epidermis samples, in order to determine the existence of a cell protection of emulsion INV1 and the existence of an improvement compared to the cell protection achieved with emulsion A. A first series of samples is subjected to the protocol without the micropollutants. A second series of samples is subjected to the protocol with the micropollutants.

TABLE 6

| | without | With | With |
|---|---|---|---|
| | application of an | application of | application of |
| | emulsion | emulsion A | emulsion INV1 |
| Without | Negative control | Internal | Internal control |
| micropollutants | | control A | INV1 |
| With | Positive control | Result A | Result INV1 |
| micropollutants | | | |

The reconstructed epidermis is that sold under the name of “Episkin™ RHE/Reconstructed Human Epidermis” by Episkin.

The micropollutants are a mixture of heavy metals and urban dust. The mixture of heavy metals consists of arsenic, cadmium, cobalt, chromium, nickel, lead, strontium and antimony. The urban dust is “Urban dust 1649b NIST® SRM®”, which corresponds to the criteria of the “National Institute for Standards and Technology” SRM 1649b.

The protocol is as follows:

The reconstructed epidermis is placed in a physiological medium

An emulsion is applied to the surface of the reconstructed epidermis in an amount of 30 microliters per sample, to form a homogeneous layer which entirely covers the reconstructed epidermis sample,

The sample is subjected to the micropollutants according to the micropollutant exposure protocol below,

The level of cell viability is then determined according to the MTT cell viability protocol below.

Micropollutant Exposure Protocol

30 microliters of emulsion are applied to each reconstructed epidermis sample. A dose of micropollutants is then applied to each sample. The samples are placed in an oven at 37° C., under an atmosphere containing 5 vol % carbon dioxide, for 24 hours. At the end of the period of exposure to micropollutants, the cell viability of each sample is determined according to the MTT protocol, repeated three times.

MTT Cell Viability Protocol

The reconstructed epidermis samples are incubated in a solution containing 1 mg/mL of “MTT”, which is 3-[4,5-dimethylthiazol-2-yl]-2,5-diphenyl tetrazolium bromide, for 3 hours at 37° C. The solution is then removed and replaced with isopropanol, and incubation is carried out for two additional hours at 22° C. Two aliquots of each reconstructed epidermis sample are transferred into each of the 96 wells of a microplate. The absorbance at 570 nm is then measured on each well with a colorimeter fitted with a microplate reader (Tecan model F200Pro).

The cell viability is calculated according to the formula: % cell viability=[absorbance of the sample at 570 nm/absorbance of the negative control at 570 nm]×100.

The absorbance values of the six samples for the cell viability measurement are presented in the following Table 7.

TABLE 7

| | Without | With | With |
|---|---|---|---|
| | application of an | application of | application of |
| | emulsion | emulsion A | emulsion INV1 |
| Without | 1.96 | 2.05 | 1.88 |
| micropollutants | | | |
| With | 0.34 | 0.69 | 0.83 |
| micropollutants | | | |

The cell viabilities of the samples are calculated from the preceding data.

TABLE 8

| | Without | With | With |
|---|---|---|---|
| | application of an | application of | application of |
| | emulsion | emulsion A | emulsion INV1 |
| Without | 100 | 104.8 | 96.1 |
| micropollutants | | | |
| With | 17.3 | 35.5 | 42.5 |
| micropollutants | | | |

In the absence of emulsion applied to the reconstructed epidermis, a cell viability of 17.3% is observed, which reflects the lethal effect of the exposure of the cells of the reconstructed epidermis to micropollutants. Application of emulsion A makes it possible to increase cell viability to 35.5%.

Emulsion INV1 according to the invention makes it possible to increase cell viability to 42.5%, that is 7% more than emulsion A, which reflects 8% better cell protection.

The film protects the explants from the penetration of micropollutants, and thus increases the number of cells which remain viable by approximately 8% compared to an emulsion according to the prior art.

### Example 3: Foundation According to the Invention

A foundation according to the invention is prepared according to the overall composition of Table 8, following the protocol below.

TABLE 8

| Phase | Trade name | INCI name | % m |
|---|---|---|---|
| A | Water | Aqua | q.s. 100 |
| A | Tabulose ® SC611 | Microcrystalline | 1.50 |
| | (Roquette) | Cellulose & Cellulose | |
| | | Gum | |
| A | Lycoat ® RS720 (Roquette) | Hydroxypropyl starch | 5.00 |
| A | Unipure White LC 985 PHY | Cl 77891 (and) Phytic | 8.30 |
| | (Sensient Cosmetic | Acid (and) Sodium | |
| | Technologies) | Hydroxide | |
| A | Unipure Yellow LC 188 PHY | Cl 77492 (and) Phytic | 1.10 |
| | (Sensient Cosmetic | Acid (and) Sodium | |
| | Technologies) | Hydroxide | |
| A | Unipure Red LC 388 PHY | Cl 77491 (and) Phytic | 0.48 |
| | (Sensient Cosmetic | Acid (and) Sodium | |
| | Technologies) | Hydroxide | |
| A | Unipure Black LC 998 | Cl 77499 (and) Phytic | 0.14 |
| | PHY(Sensient Cosmetic | Acid (and) Sodium | |
| | Technologies) | Hydroxide | |
| B | Refined oleic sunflower oil | Helianthus annuus | 5.00 |
| | (Aldivia) | seed oil | |
| B | Miglyol Coco 810(IOI OLEO | Coco- | 5.00 |
| | GmbH) | Caprylate/Caprate | |
| B | MOD (Gattefossé) | Octyldodecyl myristate | 3.00 |
| B | Montanov 202 (Seppic) | Arachidyl Alcohol (and) | 2.00 |
| | | Behenyl Alcohol (and) | |
| | | Arachidyl Glucoside | |
| B | Montanov 68 (Seppic) | Cetearyl Alcohol (and) | 2.00 |
| | | Cetearyl Glucoside | |
| C | Microcare PHC (THOR) | Phenoxyethanol & | 1.00 |
| | | Chlorphenesin | |
| C | Tocobiol C (Quimica Masso) | Tocopherol (mixed) | 0.50 |
| | | (and) Beta-Sitosterol | |
| | | (and) Squalene | |
| C | NORA SA FML00190 | Fragrance | 0.30 |
| | (MLLE PARFUMETTE/ | | |
| | ACCORDS & PARF) | | |
| D | Citric acid | Citric acid | Q.s. pH |
| | | | 5.8-6.0 |

Firstly, Tabulose SC611 is dispersed in the water required for the composition at 22° C. for 10 min by stirring with a deflocculating stirrer blade at medium speed. The pigments are then successively dispersed until a homogeneous mixture is obtained, by stirring with the deflocculating stirrer blade at medium speed. Still at 22° C., the pea starch according to the invention, Lycoat® RS720, which is a pregelatinized, hydrolyzed and hydroxypropylated pea starch, is then dispersed. Phase A obtained in this way is heated to 80° C. with gentle stirring for approximately 20 minutes.

In parallel to the preparation of phase A, phase B is prepared by weighing the required weights of ingredients of phase B and mixing them together. The whole of phase B is then heated to 80° C.

Once phases A and B are ready and at the temperature of 80° C., phase B is emulsified in phase A while stirring with the deflocculating stirrer blade at a speed of 1500-2000 rpm for 15 min. The oil-in-water emulsion obtained is then left to cool to 22° C. with slow stirring with the deflocculating stirrer blade. The preservatives and fragrance of phase C are then added, then finally the pH of the emulsion is adjusted to a value of 5.8 to 6.0 by adding citric acid diluted to 30% m in water.

Non-Transfer Test

In order to verify the non-transfer property of the foundation according to the invention, a test is carried out on a volunteer. The volunteer applies the foundation according to the invention to one cheek of their face, and a foundation not in accordance with the invention prepared according to a composition from the table without Lycoat® RS720, following the same protocol, to the other cheek. Then she waits two minutes.

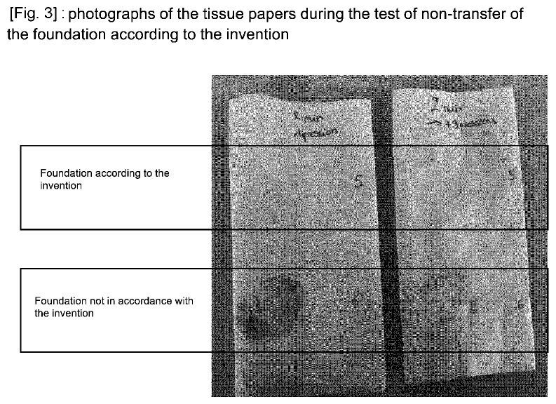

The volunteer places a tissue paper without pressure on each cheek, then applies pressure using her index finger, then she repeats this operation with a new piece of tissue paper, applying pressure nine times with the index finger. The results of this test can be seen in FIG. 3.

It is observed that the tissue paper applied to the cheek covered with the foundation not in accordance with the invention has a marked stain of foundation following the first press, which is less marked following the series of the next nine presses. Indeed, a visible amount of foundation was transferred onto the tissue paper: this foundation not in accordance with the invention does not have non-transfer properties.

Regarding the cheek covered with the foundation according to the invention, it is observed that there is no trace of foundation visible on the tissue paper, whether after the first press or following the series of nine presses. The foundation according to the invention has not transferred from the skin to the tissue paper: this foundation according to the invention has a non-transfer property.

Advantages of the Foundation According to the Invention

The foundation according to the invention has a delicate texture which enables medium to high coverage. This foundation protects from environmental pollution and offers a long-lasting uniform and matte skin color which lasts at least a whole day by virtue of the fixing by the Lycoat® RS720 starch film.

### Example 4: Mascara According to the Invention

A mascara according to the invention is prepared according to the overall composition of Table 9, following the protocol below.

TABLE 9

| Phase | Trade name | % m |
|---|---|---|
| A | Magnesium Aluminum Silicate (Magnesium | 1.5 |
| | Aluminum Silicate) | |
| A | Demineralized water | Q.s 100 |
| A | Lycoat ® RS720 (Roquette) | 10 |
| A | Polyvinylpyrrolidone | 0.20 |
| A | Hydrolyzed Collagen | 1.00 |
| A | Propylene Glycol | 5.00 |
| B | Petrolatum 3.00 | 3.00 |
| B | Petroleum Distillate (Odorless Mineral | 32.00 |
| | Spirits) | |
| B | Carnauba Wax | 5.00 |
| B | Synthetic Beeswax | 5.00 |
| B | Candelilla Wax | 3.00 |
| B | Paraffin | 2.50 |
| B | Oleamide DEA | 5.00 |
| C | Black Iron Oxide | 5.50 |
| D | Preservative | qs |

The mascara is prepared according to the following protocol.

The water is heated to 75° C., then the magnesium aluminum silicate is added with strong stirring with the deflocculating stirrer blade at 1500-2000 rpm. This stirring is maintained until a homogeneous mixture is obtained. The other constituents of phase A are then added successively, waiting until a homogeneous mixture is obtained between each addition. Phase A obtained in this way is kept at 75° C.

Separately, all the constituents of phase B are mixed with stirring, then heated to 75° C.

Phase B is then emulsified in phase A with stirring with the deflocculating stirrer blade at 1500-2000 rpm. Phase C is then added slowly with moderate stirring at 500 rpm until a homogeneous mixture is obtained. The mixture is then left to cool to 22° C. with moderate stirring, then phase D is added.

Advantages of the Mascara According to the Invention

The mascara according to the invention protects the eyelashes on which it is applied, by forming a barrier film to environmental pollution. It also has a non-transfer property, that is the mascara adheres so well to the eyelashes that it remains there when the eyelashes are rubbed with the fingers, a piece of paper, or clothing.

### Example 5: Lip Gloss According to the Invention

A long-lasting anti-pollution protective lip gloss is prepared according to the overall composition of Table 10, following the protocol below.

TABLE 10

| Phase | Trade name | INCI name | % m |
|---|---|---|---|
| A1 | Demineralized water | Aqua | q.s. 100 |
| A1 | Lycoate RS720 (Roquette) | Hydroxypropyl starch | 0.20 |
| A2 | Beauté by Roquette ® PO | Sorbitol | 60.00 |
| | 070 (Roquette) | | |
| A2 | Beauté by Roquette ® PO | Xylitol | 10.00 |
| | 370 (Roquette) | | |
| B | Keltrol (CP Kelco) | Xanthan gum | 0.30 |
| C | Peach flavor (Aromazone) | Flavor | 0.50 |
| C | FDC Yellow 6 (Sensient | Cl 15985 | q.s. |
| | Cosmetic Technologies) | | |
| C | Carmin Soluble W 3014 | Cl 75470 (and) | Q.s. |
| | (Sensient Cosmetic | Maltodextrin | |
| | Technologies) | | |
| C | Microcare PE (Thor) | Phenoxyethanol | 0.50 |

Firstly, the Lycoat® RS720 pea starch is dispersed in water at 22° C. while gently stirring with the deflocculating stirrer blade. The sorbitol Beaute by Roquette PO 070, and the xylitol Beaute by Roquette PO 370 are then added until they are completely dissolved and a homogeneous mixture is obtained, still with gentle stirring. Phase A1+A2 is obtained.

The xanthan gum is then dispersed in phase A1+A2 until a homogeneous mixture is obtained. The ingredients of phase C are then successively added into the combined mixture, with gentle stirring.

A lip gloss is thus obtained which has a syrupy texture by virtue of the xanthan gum and which, by virtue of the Lycoat® RS720, affords a smooth sensation during application and a non-tacky sensation once applied to the lips. The lip gloss forms a protective and fixative film on the lips, which protects them from environmental pollution and which enables the humectants (sorbitol and xylitol) to hydrate the lips well.

1. A use of at least one leguminous plant starch, in a preparation for topical use, as film-forming agent with a barrier effect to environmental pollution and/or with a fixative effect, wherein said starch has:
an amylose content of greater than or equal to 30%, preferentially between 30% and 75%,
and a Brookfield viscosity in aqueous dispersion at 20% by weight of solids and at 25° C. of between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s.

2. The use according to claim 1 wherein said leguminous plant starch is selected from pea starches, starches of chickpeas, broad beans, faba beans, beans, lupins, lentils, and is preferentially selected from pea starches, and most preferentially is a Pisum sativum starch.

3. The use according to claim 1, wherein said leguminous plant starch is a chemically modified leguminous plant starch, preferentially selected from hydroxyalkylated leguminous plant starches, the carboxyalkylated, the carboxymethylated, the octenylsuccinylated, the succinylated, the acetylated.

4. The use according to claim 1, wherein said leguminous plant starch is hydroxyalkylated, preferentially hydroxypropylated.

5. The use according to claim 1, wherein the hydroxyalkyl group content ranges from 0.1 to 20% by dry weight relative to the dry weight of hydroxyalkylated starch, preferentially from 1 to 10%.

6. The use according to claim 1, wherein said leguminous plant starch is selected from hydrolyzed, fluidized or dextrinized leguminous plant starches.

7. The use according to claim 1, wherein said leguminous plant starch is selected from pregelatinized or atomized leguminous plant starches.

8. The use according to claim 1, wherein the leguminous plant starch is a hydroxypropylated, hydrolyzed and pregelatinized pea starch.

9. The use according to claim 1, wherein the content by weight of film-forming leguminous plant starch with a barrier and/or fixative effect in the preparation for topical use, ranges from 0.6% to 50% by weight relative to the whole preparation for topical use, preferentially from 2% to 30% by weight, more preferentially from 5% to 15%, and most preferentially approximately 10%.

10. The use according to claim 1, wherein the barrier effect to environmental pollution makes it possible to reduce or prevent atmospheric microparticles suspended in the air and/or volatile organic compounds which are allergenic, harmful or toxic to the skin from coming into contact with the skin.

11. The use according to claim 1, wherein the barrier effect to environmental pollution makes it possible to protect cells of the epidermis and keratinocytes, preferentially makes it possible to increase cell viability of the cells of the epidermis and/or of the keratinocytes.

12. The use according to claim 1, wherein said preparation for topical use is selected from skincare products, preferentially selected from day creams, suncreams, aftersun creams, self-tanners, masks; haircare products, preferentially selected from shampoos, conditioners in cream or mask or lotion form, hairstyling products in spray or gel or wax form, dyeing products; makeup products, preferentially selected from foundations, eyeshadows, mascaras, lipsticks, pressed lip colors, lip glosses, eyeliners, nail varnishes, hygiene products, preferentially selected from cleansing gels, cleansing wipes or makeup removing wipes, or aqueous-alcoholic solutions or gels.

13. The use according to claim 1, wherein the leguminous plant starch is the only amylaceous or of amylaceous origin film-forming agent in said preparation.

14. A use of at least one hydroxyalkylated, preferentially hydroxypropylated, leguminous plant starch, in a preparation for topical use, as film-forming agent with a fixative effect, wherein said starch has:
an amylose content of greater than or equal to 30%, preferentially between 30% and 75%,
a Brookfield viscosity in aqueous dispersion at 20% by weight of solids and at 25° C. of between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s, and
a content by weight of 0.6% to 50% by weight relative to the whole preparation for topical use, preferentially of 2% to 30% by weight, more preferentially of 5% to 15%, and most preferentially of approximately 10%.

15. A use of at least one hydroxyalkylated, preferentially hydroxypropylated, leguminous plant starch, in a preparation for topical use, as film-forming agent with a barrier effect to environmental pollution, wherein said starch has:
an amylose content of greater than or equal to 30%, preferentially between 30% and 75%,
a Brookfield viscosity in aqueous dispersion at 20% by weight of solids and at 25° C. of between 10 and 10,000 mPa·s, preferentially between 20 and 5000 mPa·s, more preferentially between 50 and 1000 mPa·s, most preferentially between 75 and 500 mPa·s, and even more preferentially of approximately 150 mPa·s, and
a content by weight of 0.6% to 50% by weight relative to the whole preparation for topical use, preferentially of 2% to 30% by weight, more preferentially of 5% to 15%, and most preferentially of approximately 10%.
